(19) United States
(12) Patent Application Publication
(10) Pub. No.: US 2005/0065089 A1
(43) Pub. Date: Mar. 24, 2005

(54) TGF-beta activation and use

(76) Inventor: Bret A. Ferree, Cincinnati, OH (US)

Correspondence Address: John G. Posa;Gifford, Krass, Groh, Sprinkle,, Anderson & Citkowski, P.C., 280 N. Old Woodward Ave., Suite 400, Birmingham, MI, 48009-5394, US

(21) Appl. No.: 10/987,919

(22) Filed: Nov. 12, 2004

Related U.S. Application Data
(63) Continuation-in-part of application No. 10/426,203, filed on Apr. 29, 2003.
Application No. 10/426,203 is a continuation-in-part of application No. 09/897,000, filed on Jul. 2, 2001, now abandoned.
(60) Provisional application No. 60/215,445, filed on Jun. 30, 2000.
(60) Provisional application No. 60/519,397, filed on Nov. 12, 2003.

(51) Int. Cl. A61K 38/18

(52) U.S. Cl. 514/12

(57) ABSTRACT

Proteases are added to Platelet Rich Plasma (PRP) along with calcium and thrombin to activate latent TGF-β. The proteases include plasmin, calpin, MMP-9, thrombospondin, transglutaminase, the mannose 6-phosphate receptor (M6PR), furin, substilisin-like endoproteases, and integrins. Dermatopontin can also be added to the PRP to enhance its biologic activity.

### REFERENCE TO RELATED APPLICATION

This application claims priority from U.S. Provisional Patent Application Ser. No. 60/519,397, filed Nov. 12, 2003, and is a continuation-in-part of co-pending U.S. patent application Ser. No. 10/426,203, filed Apr. 29, 2003, which is a continuation-in-part of U.S. patent application Ser. No. 09/897,000, Jul. 2, 2001, now abandoned, which claims priority from U.S. Provisional Patent Application Ser. No. 60/215,445, filed Jun. 30, 2000. The entire content of each application is incorporated herein by reference in their entirety.

### FIELD OF THE INVENTION

This invention relates generally to the use of proteases to activate latent TGF-β in the treatment of disc disease, herniation, and other maladies.

### BACKGROUND OF THE INVENTION

Eighty-five percent of the population will experience low back pain at some point. Fortunately, the majority of people recover from their back pain with a combination of benign neglect, rest, exercise, medication, physical therapy, or chiropractic care. A small percent of the population will suffer chronic low back pain. The cost of treatment of patients with spinal disorders plus the patient's lost productivity is estimated at 25 to 100 billion dollars annually.

Seven cervical (neck), 12 thoracic, and 5 lumbar (low back) vertebrae form the normal human spine. Intervertebral discs reside between adjacent vertebra with two exceptions. First, the articulation between the first two cervical vertebrae does not contain a disc. Second, a disc lies between the last lumbar vertebra and the sacrum (a portion of the pelvis).

The spine supports the body, and protects the spinal cord and nerves. The vertebrae of the spine are also supported by ligaments, tendons, and muscles which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine.

The human intervertebral disc is an oval to kidney bean shaped structure of variable size depending on the location in the spine. The outer portion of the disc is known as the annulus fibrosis. The annulus is formed of 10 to 60 fibrous bands. The fibers in the bands alternate their direction of orientation by 30 degrees between each band. The orientation serves to control vertebral motion (one half of the bands tighten to check motion when the vertebra above or below the disc are turned in either direction).

The annulus contains the nucleus. The nucleus pulpous serves to transmit and dampen axial loads. A high water content (70-80 percent) assists the nucleus in this function. The water content has a diurnal variation. The nucleus imbibes water while a person lies recumbent. Activity squeezes fluid from the disc. Nuclear material removed from the body and placed into water will imbibe water swelling to several times its normal size. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per micro liter.

Interestingly, the adult disc is the largest avascular structure in the human body. Given the lack of vascularity, the nucleus is not exposed to the body's immune system. Most cells in the nucleus obtain their nutrition and fluid exchange through diffusion from small blood vessels in adjacent vertebra.

The disc changes with aging. As a person ages the water content of the disc falls from approximately 85 percent at birth to 70 percent in the elderly. The ratio of chondroitin sulfate to keratin sulfate decreases with age. The ratio of chondroitin 6 sulfate to chondroitin 4 sulfate increases with age. The distinction between the annulus and the nucleus decreases with age. These changes are known as disc degeneration. Generally disc degeneration is painless.

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Apart from destructive techniques, patients with herniated intervertebral discs and degenerative disc disease conservatively be treated by rest, physical therapy, oral medication, and chiropractic care. Patients that do not respond to conservative care generally undergo an injection of steroids into the epidural space of their spinal canal (epidural space) or surgery. Steroid injection reduces the inflammation surrounding herniated or degenerated discs. Decreased inflammation may reduce the pain from the disc. Unfortunately, steroid injection may hinder the healing process. Although growth factors and differentiation factors (soluble regulators) induce the healing process, it is believed that steroids may interfere with the cascade of these healing factors normally found in the body.

Given the large number of patients each year which require surgery to treat disc disease and herniation, with substantial implications in terms of the cost of medical treatment and human suffering, any solution to improve the effectiveness of non-surgical treatments would be welcomed by the medical community.

### SUMMARY OF THE INVENTION

This invention relates generally to the use of proteases to activate latent TGF-β in the treatment of disc disease, herniation, and other maladies. Calcium and thrombin are used to force platelets to degranulate so as to release latent TGF-β. In the preferred embodiment, proteases to activate the latent TGF-β. The proteases are added to the Platelet Rich Plasma (PRP), calcium, and thrombin. The proteases include plasmin, calpin, MMP-9, thrombospondin, transglutaminase, the mannose 6-phosphate receptor (M6PR), furin, substilisin-like endoproteases, and integrins. Dermatopontin can also be added to the PRP to enhance its biologic activity.

### DETAILED DESCRIPTION OF THE INVENTION

In my U.S. patent application Ser. Nos. 09/897,000 and 10/426,203, the entire content of each being incorporated herein by reference, I teach the use of soluble regulators such as growth factors and differentiation factors to treat disc disease and herniation. Such substances may be produced with recombinant genetic techniques, or obtained from animal sources. In the preferred embodiment, the materials are concentrated from a patient's blood and injected into the epidural space of the spinal canal and or the intervertebral disc using techniques well known to those skilled in the art.

I also describe the use of calcium and thrombin to force the platelets to degranulate so as to release latent TGF-β. The blood is centrifuged to obtain platelets, and the platelets release the soluble regulators/growth factors by adding a mixture of calcium chloride and topical bovine thrombin. According to one example, 6 ml of platelet rich plasma is combined with 1 ml of the calcium chloride-thrombin mixture and injected into the disc or spinal canal. Alternatively, the platelet rich plasma and calcium chloride-thrombin mixture may be injected separately. Soluble regulators obtained from other sources or different amounts of the platelet rich plasma than described above could also be used.

This invention adds the use of proteases to activate the latent TGF-β. The proteases are added to the Platelet Rich Plasma (PRP), calcium and/or thrombin. The proteases include plasmin, calpin, MMP-9, thrombospondin, transglutaminase, the mannose 6-phosphate receptor (M6PR), furin, substilisin-like endoproteases, and integrins. Dermatopontin can also be added to the PRP to enhance its biologic activity.

1. A method of activating latent TGF-β, comprising the steps of:
providing platelet-rich plasma (PRP); and
using one or more proteases to activate latent TGF-β in the PRP.

2. The method of claim 1, wherein the proteases include plasmin, calpin, MMP-9, thrombospondin, transglutaminase, the mannose 6-phosphate receptor (M6PR), furin, substilisin-like endoproteases, and integrins.

3. The method of claim 1, including the step of adding the proteases to a combination of platelet rich plasma (PRP), calcium and/or thrombin.

4. The method of claim 1, further including the step of adding dermatopontin to enhance its biologic activity.

5. The method of claim 1, further including the step of using the activated TGF-β as part of a medical procedure.

6. A method of activating latent TGF-β, comprising the steps of:
providing platelet-rich plasma (PRP); and
using of calcium and/or thrombin to force the platelets to degranulate so as to release latent TGF-β; and
using one or more proteases to activate the latent TGF-β.

7. The method of claim 6, wherein the proteases include plasmin, calpin, MMP-9, thrombospondin, transglutaminase, the mannose 6-phosphate receptor (M6PR), furin, substilisin-like endoproteases, and integrins.

8. The method of claim 6, further including the step of adding dermatopontin to enhance its biologic activity.

9. The method of claim 6, further including the step of using the activated TGF-β as part of a medical procedure.

10. A non-surgical approach to treating disc disease and herniation, comprising the steps of:
providing platelet-rich plasma (PRP);
using one or more proteases to activate latent TGF-β in the PRP; and
delivering the PRP with the activated TGF-β to a spinal region.

11. The method of claim 10, wherein the proteases include plasmin, calpin, MMP-9, thrombospondin, transglutaminase, the mannose 6-phosphate receptor (M6PR), furin, substilisin-like endoproteases, and integrins.

12. The method of claim 10, including the step of adding the proteases to a combination of platelet rich plasma (PRP), calcium and/or thrombin.

13. The method of claim 1, further including the step of adding dermatopontin to enhance its biologic activity.
